(19) United States
(12) Patent Application Publication
(10) Pub. No.: US 2026/0098055 A1
(43) Pub. Date: Apr. 9, 2026

(54) REMOVAL OF POLYMERS FROM A SOLUTION BY USING MACROCYLCES AND/OR HYDROPHOBIC PARTICLES

(71) Applicant: Cube Biotech GmbH, Monheim (DE)

(72) Inventors: Barbara Maertens, Köln (DE); Jan Kubicek, Köln (DE); Roland Fabis, Leverkusen (DE); Philipp Timo Hanisch, Langenfeld (DE); Sergej Balanda, Düsseldorf (DE); Michael Erkelenz, Solingen (DE); Nina Valeska Heckmann, Langenfeld (DE)

(21) Appl. No.: 19/112,126

(22) PCT Filed: Sep. 7, 2023
(86) PCT No.: PCT/EP2023/074566

§ 371 Date: Mar. 14, 2025

Related U.S. Application Data
(60) Provisional application No. 63/408,637, filed on Sep. 21, 2022.

(51) Int. Cl. C07K 1/14

(52) U.S. Cl. CPC C07K 1/145

(57) ABSTRACT

A method for removing free polymers present in a solution containing a hydrophobic protein, wherein the method comprises (a) contacting the solution containing the free polymers not bound to the hydrophobic protein, with a macrocycle and/or a hydrophobic particle so that the free polymers bind to the macrocycle and/or hydrophobic particle to obtain a complex of said polymers and the macrocycle and/or hydrophobic particle, and (b) removing the complex of polymer and the macrocycle and/or hydrophobic protein from the solution. Macrocycles and a kit containing a macrocycle and/or hydrophobic particle to be employed in the method.

### FIELD OF THE INVENTION

The present invention relates to a method for removing of free polymers from a solution employing macrocycles like cyclodextrins, calixarenes, curcurbituryl or pillararene, and/or hydrophobic particles, such as butyl agarose, the use of these materials in such a method, a composition comprising a material adapted for this method, and a kit containing macrocylces and/or hydrophobic particles for removing free polymers from a solution.

### BACKGROUND OF THE INVENTION

The solubilisation, stabilization and purification of membrane proteins out of the native membrane surrounding is a well-established procedure. It is dependent on a number of parameters. Most parameters can be optimized during the purification process to a higher efficiency. A crucial step in the purification process is the effective solubilization of the target protein out of the cell membrane, which is achieved by employing polymers.

To effectively solubilize maximum amounts of membrane protein the employed polymer has to be used in excess. However, not all employed polymer is used during the solubilization process leading to free polymer in the purified solubilization supernatant. Due to the chemical properties (unspecific interaction of the polymers, interference in downstream processes), the free polymer causes drawbacks. On the one hand, the protein binding efficiency during affinity chromatography is influenced. Free copolymer massively interferes during the protein binding step to many different resins leading to massive losses during protein-resin binding. Further downstream processing is influenced as well. Due to its chemical properties the free polymer massively interferes with several downstream processes like mass spectrometric analysis and enzyme kinetic analysis or SDS-PAGE.

### BRIEF SUMMARY OF THE INVENTION

Therefore, the technical problem underlying the present invention is to provide a method, a use, a composition and a kit with which the free polymer can be removed from a purified solubilisation supernatant so that the above drawbacks can be avoided.

In one aspect, the present invention relates to a method for removing free polymers present in a solution containing a hydrophobic protein, which includes: (a) contacting the solution containing the free polymers not bound to the hydrophobic protein, with a macrocycle and/or a hydrophobic particle so that the free polymers bind to the macrocycle and/or hydrophobic particle to obtain a complex of said polymers and the macrocycle and/or hydrophobic particle, and (b) removing the complex of polymer and the macrocycle and/or hydrophobic protein from the solution.

In a related aspect of the invention a kit for carrying out a method for removing free polymers present in a solution containing a hydrophobic protein is provided, which includes a macrocycle and/or a hydrophobic particle, and instructions for carrying out the method for removing the polymer.

### BRIEF DESCRIPTION OF THE DRAWINGS

In the following, the present invention is further illustrated by referring to the examples and the figures. These examples shall not be construed to limit the invention thereto.

FIG. 1 shows the removal of a free polymer using a cyclodextrin bound to magnetic beads.

FIG. 2 shows a further removal of a free polymer using a cyclodextrin bound to magnetic beads.

FIG. 3 shows a further removal of a free polymer using a cyclodextrin bound to magnetic beads.

FIG. 4 shows a further removal of a free polymer using a cyclodextrin bound to magnetic beads.

FIG. 5 shows the removal of a free polymer using a cyclodextrin bound to agarose beads.

FIG. 6A-C shows the comparison of free copolymer reduction utilising Cyclodextrin coupled agarose vs Size Exclusion chromatography.

FIG. 7 shows the results of the removal of polymers from a cell lysis solution containing complexed and solubilized membrane proteins.

### DESCRIPTION OF THE INVENTION

It has been found that free polymers present in a solution containing a hydrophobic protein can be removed by a method, comprising:
(a) contacting the solution containing the free polymers not bound to the hydrophobic protein, with a macrocycle and/or hydrophobic particle so that the free polymers bind to the macrocycle and/or hydrophobic particle to obtain a complex of said polymers and the macrocycle and/or hydrophobic particle, and
(b) removing the complex of polymer and the macrocycle and/or hydrophobic particle from the solution.

As understood according to the present invention, the term “hydrophobic protein” means integral membrane proteins, peripher membrane proteins or proteins with exposed hydrophobic regions, e.g. proteins with amphiphatic helices, containing GTpases, ATG proteins, proteins containing the ENTH/ANTH or a Bar domain as well as their interacting proteins. An overview can be found at Mikhail A Zhukovsky, Angela Filograna, Alberto Luini, Daniela Corda, Carmen Valente; Protein Amphipathic Helix Insertion: A Mechanism to Induce Membrane Fission; Front Cell Dev Biol. 2019 December 10; 7:291. doi: 10.3389/fcell.2019.00291. eCollection 2019.

The free polymer to be removed according to the present invention can stem from the solubilisation, stabilization and optionally purification of membrane proteins out of the native membrane surrounding. This is a well-established procedure. The skilled person knows methods and materials for carrying it out. Briefly, one step in this process is the effective solubilization of the target protein out of the cell membrane, which is achieved by employing certain polymer known in this technical field. To effectively solubilize maximum amounts of membrane protein the employed polymer has to be used in excess. However, not all employed polymer is used during the solubilization process leading to free polymer in the purified solubilization supernatant.

The polymer used in this process can be a homopolymer or a copolymer, as will be specified in more detail in the following.

The term “removing” as used herein means that the amount of the free polymer is reduced after carrying out the method according to the present invention compared to the amount of free polymer before the method according to the present invention is carried out.

With the method according to the present invention, it is possible to remove the polymer from a solution rapidly without any effort in a highly efficient manner. Furthermore, due to removing the polymer, they do not disturb the down streaming processing, like mass spectrometry and enzyme kinetics, of the remaining components of the solution, for example the membrane proteins. It is possible to remove the polymer to 95% or even more.

In the following, a general protocol for purification of a membrane protein stabilized in copolymer (e.g. AASTY (Copolymers from styrene and acrylic acid), Ultrasolute Amphipol (polyacrylic acid, partially coupled to amides by cycloalkyl amines or cycloalkyl alkylamines)) exemplified:

As explained above, the solubilisation, stabilization and purification of membrane proteins out of the native membrane surrounding is dependent on a number of parameters. Most parameters can be optimized during the purification process to a higher efficiency. The parameters include buffer conditions (for example salt, pH), choice of polymer, protein-to-solubilisation agent-ratio, temperature, and time. First, cell lysis and centrifugation are carried out by for example using the following parameters: Adding of protease inhibitors (PI) to buffer and readjust pH value then disrupting cells (e.g., Sonification, French Press). centrifugation at 9 000 rcf for 30 min at 4° C., discarding pellet (cell debris), collecting supernatant, centrifugation of the supernatant at 100 000 rcf for 1 h at 4° C., discarding supernatant and homogenize pellet. Then the solubilisation of membrane proteins is carried out: Polymers form a synthetic nanodisc around the protein, thereby maintaining the native phospholipid environment and preserving the native and thus functional properties of the protein in a convenient one step manner (solubilization and stabilization). Detergents on the other hand form micelles around the hydrophobic belt, thus remove the lipids from the surrounding. For native condition the unique lipid environment needs to be conserved.

In one embodiment, the hydrophobic protein is a membrane protein, in particular a membrane associated protein or an integral membrane protein. Examples of the membrane protein can be selected from the group consisting of membrane receptor proteins, membrane enzymes, cell adhesion proteins, and transporter proteins, such as ABC transporters, ion channel proteins, water channel proteins (aquaporins), membrane-based ATPases, SLC transporters. That is, as a starting material for the method according to the present invention, a solution of the free polymer is used which stems from the solubilisation, stabilisation and purification of the above-mentioned membrane proteins out of their native surrounding by employing a polymer.

As understood according to the present invention, a macrocycle is a molecule containing a ring of at least 12 atoms/ions. Furthermore, as understood according to the present invention a hydrophobic particle is a particle consisting of a solid phase and a surface with hydrophobic groups. According to the definition in Wikipedia, hydrophobic materials are water-repelling. Examples for these groups are linear and branched alkyls, aromatic groups such as phenyl, methylphenyl, ethylphenyl, styrene, polyacrylate and methacrylate, fatty acid esters and poplypropylene glycol. It is pointed out that the hydrophobic particle is different from the hydrophobic protein, i.e., it is understood that the hydrophobic protein is not to be considered as a hydrophobic particle. The hydrophobic particle can be a hydrophobic resin, from such materials such as polystyrene, polyacrylate, polymethacrylate, and can be a composite material from one or more hydrophobic substance, and a hydrophilic material, such as silica, a metal oxide, a polysaccharide, such as dextrane or agarose. In one embodiment, the macrocycle is selected from the group consisting of cyclodextrins, calixarenes, curcurbituryls and pillararenes. Example of the hydrophobic particle is butyl agarose.

In a further embodiment, the cyclodextrin is selected from the group consisting of α-cyclodextrin, β-cyclodextrin, γ-cyclodextrin and δ-cyclodextrin or a mixture of at least two of said cyclodextrins. The cyclodextrins are able to reversible bind free copolymer out of a solution, which can be accomplished by a concentration dependent, gradual binding. The polymers can react different to the cyclodextrin depending on their unique chemical properties so that by routine testing the suitable cyclodextrin can be chosen for removing the polymer.

In one embodiment, the macrocylce is provided on magnetic beads or agarose beads. This shows a significant effect on polymer binding.

Magnetic beads, which can be used, contain a magnetic core made of magnetite which is covered in different materials. Magnetic beads are typically either ferri (or ferro-) magnetic, or superparamagnetic. Ferri/ferromagnetic magnetic cores are typically large (>30 nm) and show a strong magnetic moment. They retain this magnetic moment even after removal of the magnetic field. This effect is called “magnetic remanence”. The strong magnetic field leads to a fast separation of the beads in the magnetic field. At the same time, they sometimes show self-magnetism and may attach to metal surfaces. Superparamagnetic magnetic cores are smaller (5-30 nm), and their magnetic moment is weaker. When the outer magnetic field is removed, the beads lose their magnetism. At the same time, use with metal surfaces is facilitated.

Alternative embodiments include functionalized membranes and so-called monolithic columns. For instance, cellulose membranes can be chemically modified with cyclodextrins, hydrophobic polymers, linear and branched alkyl, or aromatic molecules in order to prepare a functionalized membrane, which holds back the unbound copolymers. The chemistry used for this modification is almost identical to the methods described for agarose and magnetic beads (see below).

Porous monolithic columns have been developed by Frechet and Svec, who polymerized styrene and (meth)acrylates in presence of a porogen. A silica monolith is the solid continuous block of porous material with bimodal distribution dimensions of pores (macropores and mesopores). Mor details are given in: Unger KK, Skudas R, Schulte MM. Particle packed columns and monolithic columns in high-performance liquid chromatography-comparison and critical appraisal. J Chromatogr A 2008; 1184:393-415.

The synthesis of cyclodextrin-modified agarose and magnetic beads can be performed in the following manner, but is not limited to:
Epoxy-activated agarose or magbeads are reacted with cyclodextrin (variant A). This synthesis variant is described in Xiangling He, Tianwei Tan, Bingze Xu, Jan-Christer Janson, Separation and purification of puerarin using cyclodextrin-coupled agarose gel media, Journal of Chromatography A, 1022 (2004) 77-82.
NHS-activated agarose or magbeads is reacted with amino-cyclodextrin (variant B). This synthesis variant is described in Trung Nguyen, Neel S. Joshi, and Matthew B. Francis, An Affinity-Based Method for the Purification of Fluorescently-Labeled Biomolecules, Bioconjugate Chem. 2006, 17, 869-872.

Both synthesis variants lead to cyclodextrin-modified particles, which effectively remove the macromolecules according to this patent application.

In particular, the magnetic beads can have the following properties to provide very suitable results: Medium sized beads (20-40 μm) with a ferrimagnetic core, and agarose coating: For high polymer binding, low unspecific binding, and efficient separation; and large or extra-large magnetic agarose beads (70-120 μm or up to 1000 μm).

In one embodiment, the cyclodextrin is provided in cross-linked form. These cyclodextrin particles also allow the removal of copolymers.

Cross-linking of cyclodextrines can be done with reagents like epichlorohydrine, diepoxides, carbonyl diimidazole of divinylsulfone. Examples for the preparation of cross-linked carbon hydrates can be found at Xiangling He, Tianwei Tan, Bingze Xu, Jan-Christer Janson, Separation and purification of puerarin using-cyclodextrin-coupled agarose gel media, Journal of Chromatography A, 1022 (2004) 77-82.

Other materials, which can be used, are calixarenes, cucurbiturils, pillararenes, in cross-linked form or bound to solid phase.

Also, agarose in concentrations of 6%, to 12%, dextrans in cross-linked form or bound to other polymer particles, copolymers from methylene-bis-acrylamide and dextran, which can be plain (non-modified) or modified with hydrophobic groups, such as linear and branched alkyl, alkenyl, alkinyl, and aromatic, are effective in reducing the concentration of the polymers in protein solutions.

Organic polymer particles, such as Biobeads SM-2 (polystyrene particles, Bio-Rad Inc., Hercules, CA, USA), Macro-Prep Methyl or Butyl HIC resin (polymethacrylate particles, Bio-Rad Inc.) or the like allow a reduction of the polymers.

Calixarenes are phenol molecules, connected by methylene or other functional groups. So, for instance, in the work of Aseyev (Wiktorowicz, H. Tenhu and V. Aseyev, Polym. Chem., 2013, 4, 2898) they are functionalized with tetraethylene glycol or alkyl groups in order to tailor the polarity of the molecules. These molecules can be coupled to solid phase with standard methods known by a person skilled in the art, for instance described for cyclodextrines.

Cucurbiturils were first synthesized by Behrend in 1905 from acid-catalyzed condensation reactions of urea, glyoxal, or formaldehyde. They can interact noncovalently with various sizes of positively charged/neutral guests to form supramolecular host-guest complexations via hydrogen bonding, charge-dipole, and the hydrophobic/hydrophilic effect.

Cucurbituril [6] can form inclusion complexes with hydrophobic neutral guests (tetrahydrofuran and benzene), protonated amines and p-methylbenzylamine, while Cucurbituril [7] can interact with naphthalene, protonated adamantanamine, and carborane, respectively.

Cucurbituril [8], with a large cavity, is involved in the complexation with large-sized guests (cyclen, cyclam, and their metal complexes). Here the synthesis of supramolecular hydrogels from a CB[8]-threaded highly branched polymer (HBP-CB[8]) and linear hydroxyethyl cellulose-functionalized naphthalene is described by C.S. Y. Tan, J. Liu, A.S. Groombridge, S.J. Barrow, C.A. Dreiss and O.A. Scherman, Adv. Funct. Mater., 2018, 28, 1702994; This procedure can also be used to prepare cucurbituril-modified particles.

Pillararenes have a pillar-shaped structure by methylene bridges at the para positions of functionalized aromatic rings. This structure makes them very effective in binding with electron-withdrawing or neutral guests. The good solubility in both organic and aqueous solutions gives them a broad applicability.

These molecules, prepared by e.g. a reaction of 1,4-dimethoxybenzene with paraformaldehyde and lewis acids (T. Ogoshi, S. Kanai, S. Fujinami, T.A. Yamagishi and Y. Nakamoto, J. Am. Chem. Soc., 2008, 130, 5022-5023) can be used to selectively bind copolymers from a solution.

Agarose, dextran polymers, dextran-functionalized agarose, copolymers from methylene-bis-acrylamide and dextran, or the like can be modified by hydrophobic groups like butyl, phenyl and octyl by a reaction of the polysaccharide with butyl, phenyl or octyl glycidyl ester under lewis catalysis with e.g. boron trifluoride diethyl etherate under anhydrous conditions. This procedure is described in Feng Qing-zheng; Meng Qing-qiang; Wang Jia-xing; Ma Guang-hui; Ma Run-yu; Su Zhi-guo, Preparation of Butyl-agarose Chromatography Media with Controlled Ligand Density through Direct Coupling Reaction, The Chinese Journal of Process Engineering, 2006, 6(6): 959-963. Butyl, Phenyl and Octyl Agarose can be purchased at Cube Biotech, Monheim Germany.

The particles, membranes and monolithic columns suitable for removing the copolymers can be applied in a gravity flow column, in a FPLC column, in medium pressure chromatography and in a HPLC column. These columns can be used with automated chromatography systems, like Äkta (Cytiva) or BioLogic™ Low-Pressure Liquid Chromatography Systems (Bio-Rad).

In one embodiment, the macrocycle and/or hydrophobic particle is washed with the solvent, which is the solvent of the solution of the polymer, before step (a) is carried out. In a still further embodiment, step (a) and step (b) are repeated at least once, for example 1 to 3 times so that steps (a) and (b) are carried out 1 to 4 times in total. By repeating step (a) and step (b), more free protein can be removed than by carrying out them only once. This makes it possible to adjust the removal according to the needs of the downstream processing.

As pointed out above, the polymer can be a homo polymer or a copolymer. In one embodiment, the polymer can have hydrophilic groups, such as COOH, maleimide, OH, amines, ammonium salts, zwitter ions like phosphocholines, and hydrophobic groups, such as polymerized styrene groups, polymerized diisobutylene groups, or linear C1 to C16 (like methyl and ethyl) aliphatic groups, branched C1 to C16 (like isopropyl or t-butyl) aliphatic groups and cyclic C5 to C12 aliphatic or aromatic groups.

The molecular weight of the polymer employed according to the method of the present invention can be 1900 to 20000, for example 2000 to 18000, or 2000 to 15000, or 4000 to 16000, or 4000 to 13000 or 5000 to 14000. The molecular weight can be measured by gel permeation chromatography or mass spectrometry.

Examples for the polymers can be, but are not limited to styrene/maleic acid copolymers, sold by the trade name “SMA”, derivatives of styrene/maleic acid copolymers like SMA 200 and 300, styrene/maleimide copolymers, like SMA 502. These substances can also be functionalized on the COOH groups, with amines, like ethanol amine or ethylene diamine to amides, or with alcohols like glycerol to esters. The polymers can also be functionalized with polyethylene glycols to esters and with aminated polyethylene glycols to amides.

The polymer can be diisobutylidene/maleic acid copolymers, for example DIBMA 10 and DIBMA 12 from Cube Biotech, derivatives of diisobutylidene/maleic acid copolymers, like DIBMA Gly, DIBMA Glu, Glyco DIBMA, and diisobutylidene/maleimide copolymers. DIBMA coplymers can be functionalized with the same molecules like SMA.

Further polymers can be copolymers from styrene and acrylic acid, in particular with a molecular weight of 5.500 and 11.000 and a relation acrylic acid/styrene of 45%/55% to 55%/45%, sold under the name “AASTY”.

Modified polymers from polyacrylic acid can be used, where 10-90% of the carboxylic acid groups can be modified to amides with cyclooctylamine, 2-cyclohexyl-ethylamine, and the like, in some embodyments with substances like DCC (dicyclohexyl carbodiimide), EDC [1-Ethyl-3-(3-dimethylaminopropyl)carbodiimide], NHS (N-hydroxy succinimide), or PyBOP, HBTU or TBTU. These substances are sold under the name “Amphipol Ultrasolve.”

In addition to the above disclosure of the polymer, in the following a further description of the polymer is given.

Polymers with hydrophilic and hydrophobic functional groups:

Examples for hydrophilic groups could be, but are not limited, to polymers of acrylic acid and methacrylic acid, maleic acid, carboxylic acid groups in general, amides with α,ω alkylene diamine, ω-hydroxyalkyl amine and ω-aminoalylthiols, trimethylammonio-alkylamin, amide from carboxylic acid groups with amino-glycerol TRIS, or Bis-Tris, amide with maltosamine, glucosamine, mannosamine and other amino-functionalized carbo hydrates, taurin.

Also, esters of carboxylic acid groups with polyethylene glycols, diols, triols, polyols, and carbo hydrates can be mentioned.

Other examples can be maleimides, with the nitrogen atom functionalized with alkyl chains with alcohol, thiol, amine, ammonium salts and the like.

Alternatively, zwitterionic molecules, consisting of ammonium and phosphate groups, ca be linked onto carboxylic groups, like it is described in US2020281855A1 or US2021171673A1.

Examples for hydrophobic groups could be, but are not limited, to polymerized styrene and derivatives, such as methylstyrene, diisobutylene and linear and branched alkanes, like 2-propyl, hexyl, octyl, or decyl, coupled to carboxylic groups via ester or amide functions. Also, maleimide groups with alkyl or aryl groups on the amino function are suited examples.

An example for the synthesis of styrene-maleic acid copolymers can be seen in Shintaro Sugai, Nobumichi Ohno, Conformational transitions of the hydrophobic polyacids, Biophysical Chemistry, Volume 11, Issues 3-4, June 1980, Pages 387-395.

The use of SMA for building a complex with lipids is described in WO 2006/129127 and references therein.

SMA can be purchased at Orbiscope or Cube Biotech, as SMALP 140, SMALP 200, or SMALP 300. The synthesis of copolymers from diisobutylene and maleic acid anhydride is described in U.S. Pat. No. 4,250,289 by BASF. Hydrolysis of anhydride copolymer to diisobutylene-co-maleic acid is described in Lee, Nature Protocols Vol. 11, No. 7, 2016, 1149-1162, which is described for SMA copolymer, but can be applied to DIBMA without problem.

The synthesis of a DIBMA polymer with a functionalization of a glucosamine on 50% of all carboxy groups can be found on: Bartholomäus Danielczak, Marie Rasche, Julia Lenz, Eugenio Pérez Patallo, Sophie Weyrauch, Florian Mahler, Michael Tope Agbadaola, Annette Meister, Jonathan Oyebamiji Babalola, Carolyn Vargas, Cenek Kolar and Sandro Keller, A bioinspired glycopolymer for capturing membrane proteins in native-like lipid-bilayer nanodiscs, DOI: 10.1039/D1NR03811G (Paper) Nanoscale, 2022, 14, 1855-1867.

DIBMA can be purchased at Cube Biotech as DIBMA 10 and DIBMA 12.

The preparation of poly(acrylic acid-co-styrene) copolymers is described in WO 2020 257637 and Simon Harrisson, Francesca Ercole and Benjamin W. Muir, Living spontaneous gradient copolymers of acrylic acid and styrene: one-pot synthesis of pH-responsive amphiphiles, Polym. Chem., 2010, 1, 326-332.

Sometimes the copolymer is a copolymer from styrene and acrylic acid, or a copolymer from styrene and an acrylic acid derivative. Any copolymer derivative may find use in the subject copolymers. Examples for derivatives are acrylates, methacrylates, acrylic esters, acrylamides, and N-substituted acrylamides. In certain cases, the acrylic esters or acrylamides are substituted with a zwitterionic species, as described in US patent application 20190062469A1, the disclosure of which is incorporated herein by reference.

In certain embodiments the copolymer contains acrylic acid or an acrylic acid derivative content of from 20% to 80%, 30% to 70%, 35 to 65%, or 40 to 60%.

The synthesis of Amphipol is described in WO 115083 and in Marconnet, A., Michon, B., Le Bon, C., Giusti, F., Tribet, C., & Zoonens, M. (2020). Solubilization and stabilization of membrane proteins by cycloalkane-modified amphiphilic polymers. Biomacromolecules. doi: 10.1021/acs.biomac.0c00929.

Additional polyacrylates, modified with alkyl groups like pentyl, hexyl, and tert-butyl, are described in US 2020/0383918.

Polymethacrylate, containing butyl methacrylate (BMA) in Copolymer: ˜0.52 and methyl acryoloxy choline (MAC) in Copolymer: ˜0.48, with a degree of polymerization (DP): ˜39.00, is distributed by Avanti Polar Lipids, with the brand name Polymethacrylate Copolymer (N-C4-52-6.9). Other polymethacrylates are described in Yasuhara K, Arakida J, Ravula T, Ramadugu SK, Sahoo B, Kikuchi JI, Ramamoorthy A. 2017. Spontaneous Lipid Nanodisc Fomation by Amphiphilic Polymethacrylate Copolymers. J Am Chem Soc. 139(51): 18657-18663.

Polyacrylate polymers, modified with alkanes, such as n-butyl, t-butyl, pentyl, neopentyl, and hexyl are described in Nathaniel Z. Hardin, Thirupathi Ravula, Giacomo Di Mauro, Ayyalusamy Ramamoorthy, Hydrophobic Functionalization of Polyacrylic Acid as a Versatile Platform for the Development of Polymer Lipid Nanodiscs, Small. 2019 March; 15(9): e1804813. doi: 10.1002/smll.201804813, and US2020383918A1.

Alternatively, linear polysaccharides with a polymerization degree of less than 100, functionalized with hydrophobic groups, are mentioned in US2022 093587A. Examples for linear carbo hydrates are inulin, and examples for hydrophobic groups are alkyl, alkenyl, alkynyl, cycloalkyl, or heteroalkyl having 1-3 hetero atoms. The hydrophobic group is bound to the carbo hydrate via an ether, ester, or amide group.

In one embodiment, step (a) of the method according to the present invention is carried out 1 minute or less. Longer incubation times have no significant enhancing effect. That is, a very fast method for removing the polymer from the solution is provided by the method according to the present invention.

In the following, a general description of the method according to the present invention is given to illustrate it in more detail: To remove up to 50% of free polymer from a solution an equal volume of MagBead slurry (25%, v/v) can be used.

1 step reduction: One sample tube with for example 50 μl MagBead slurry is prepared. The storage buffer is removed. Washing with sample buffer is carried out, for example two times, and the sample buffer is removed. Sample is added to this first sample tube, for example 50 μl, the mixture is incubated for example about 1 min and the sample is removed.

Utilizing a 4 step removal process results in a reduction up to 95% of the free copolymer. Four sample tubes with for example 50 μl MagBead slurry each are prepared. The storage buffer is removed. Washing with sample buffer is carried out, for example two times, and the sample buffer is removed. The sample, for example 50 μl, is added to the first sample tube, the mixture is incubated for example 1 min, the sample is removed and added to the next sample tube followed by the incubation of for example about 1 minute. This is repeated two more times.

The present invention further provides the use of a cyclodextrin in the method according to the present invention. The employed cyclodextrin as well as the details of the method are described above so that it its referred to the above description in its entirety.

Furthermore, the present invention provides a composition comprising a cyclodextrin bound on a carrier for carrying out the above described as well as a kit for carrying out this method. For the description of the materials and the process steps of the method, it is referred to the above detailed description.

### Example 1: Removal of Free Copolymer from Small Sample Volumes Using CD (Cyclodextrin) Coupled Magnetic Beads

To remove up to 50 % by weight of free copolymer from a solution an equal volume of magnetic beads (MagBead from Cube biotech GmbH as slurry (25%) used.

### 1 Step Reduction:

Prepare 1 sample tubes with 50 μl MagBead slurry each. Remove storage buffer. Wash twice with sample buffer. Remove sample buffer. Add 50 μl sample to first sample tube-incubate 1 min remove sample

Utilizing a 4 step removal process results in a reduction up to 95% of the free copolymer.

### 4 Step Reduction:

Prepare 4 sample tubes with 50 μl MagBead slurry each. Remove storage buffer. Wash twice with sample buffer. Remove sample buffer. Add 50 μl sample to first sample tube-incubate 1 min remove sample and add to next sample tube-incubate 1 min-repeat two more times. Use sample in assay of choice.

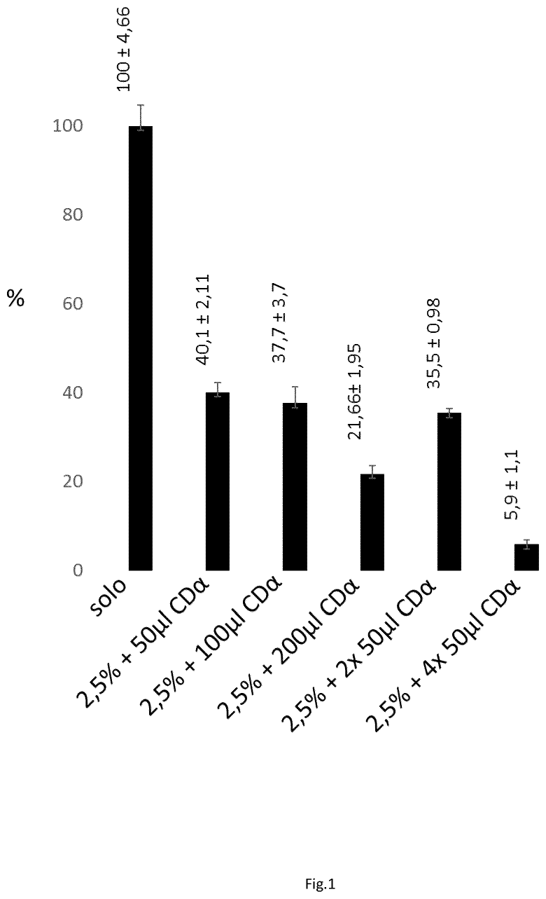

In the following, the experimental details of FIG. 1 to 4 and the results obtained are explained.

### FIG. 1

In the experiment relating to FIG. 1, 50 μl of 2.5% SMA200 solution was incubated with different volumes of CDα coupled magnetic beads for 1 min resulting in a reduction capacity of up to 95% (utilizing a 4-step removal process). A 4-step removal process results in a higher polymer reduction compared to a 1 step removal process with similar magnetic bead volume. 2.5% SMA200 solution set as 100% (solo). (CDα=Cyclodextrin alpha coupled magnetic beads; 50 μl 25% slurry=12.5 μl pure CDα volume)

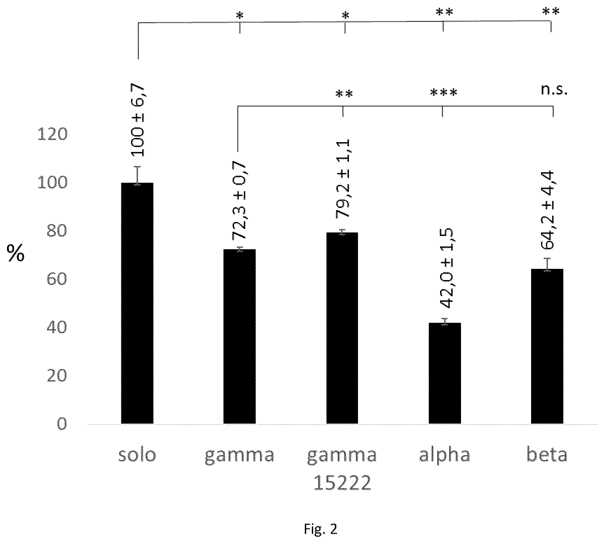

### FIG. 2

In the experiment relating to FIG. 2, 50 μl of 1.25% AASTY11-45 solution was incubated with identical volumes (50 μl of 25% slurry) of different CD coupled magnetic beads for 1 min (solo=without magnetic beads; gamma=CDγ coupled magnetic beads; gamma 15222=CDγ coupled magnetic beads; alpha=CDα coupled magnetic beads; beta=CDβ coupled magnetic beads) resulting in a reduction capacity of up to 60% (utilizing a 1 step removal process). AASTY is the only polymer of all tested which shows significant enhanced binding to CDα. 1.25% AASTY11-45 solution set as 100%. (50 μl 25% slurry=12.5 μl pure CDα volume).

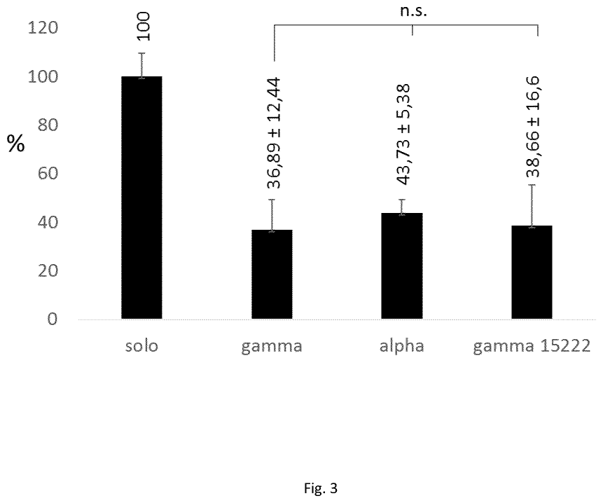

### FIG. 3

In the experiment relating to FIG. 3, 50 μl of 1.25% DIBMA12 solution was incubated with identical volumes (50 μl of 25% slurry) of different CD coupled magnetic beads for 1 min (solo=without magnetic beads; gamma=CDγ coupled magnetic beads; gamma 15222=CDγ coupled magnetic beads; alpha=CDα coupled magnetic beads) resulting in a reduction capacity of up to 60% (utilizing a 1 step removal process). DIBMA12 does not show significant enhanced binding to CDα compared to CDγ. 1.25% DIBMA12 solution set as 100%. (50 μl 25% slurry=12.5 μl pure CDα volume).

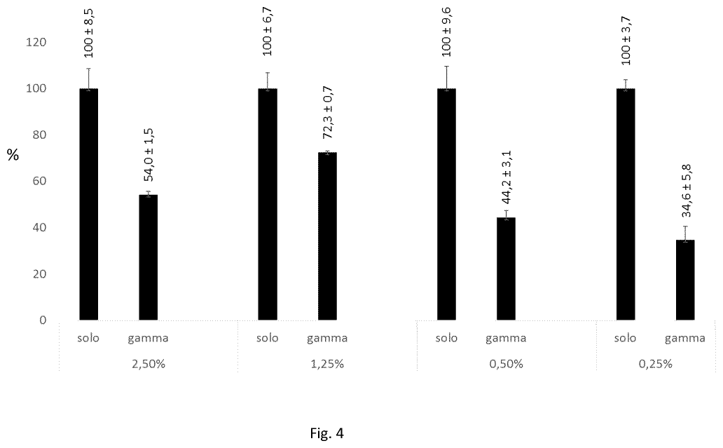

### FIG. 4

In the experiment relating to FIG. 4, 50 μl of different concentrated AASTY11-45 solutions (2.5%; 1.25%; 0.5%; 0.25%) were incubated with identical volumes (50 μl of 25% slurry) of CDγ coupled magnetic beads for 1 min (solo=without magnetic beads). Similar volumes of CDγ coupled magnetic beads do not necessarily result in an enhanced reduction capacity utilizing a 1 step removal process. Using a 10 times diluted AASTY11-45 sample (0.25%) results in a 65.4% reduction while a 2.5% AASTY11-45 sample is reduced by 46% utilizing a similar volume of CDγ magnetic beads while a 1.25% AASTY11-45 sample is only reduced by 27.7%. AASTY11-45 solution set individually as 100% (from left to right: 2.5%; 1.25%; 0.5%; 0.25%). (50 μl 25% slurry=12.5 μl pure CDα volume).

### Example 2: Removal of Free Copolymer from Large Sample Volumes Using CD Coupled Agarose

To remove up to 90 % of free copolymer from a solution an equal volume of agarose (double volume of 50% slurry e.g., 5 ml copolymer solution and 10 ml agarose slurry) is used. A higher agarose bed ensures effective remo. val of the free copolymer.

### 1 Step Reduction:

Prepare a fitting column with agarose slurry (e.g., 10 ml 50% agarose slurry in a 1 cm diameter column for 5 ml of copolymer solution). Let the storage solution drop out via gravity flow. Wash agarose with 5 column volume (CV) of sample buffer. Add sample and let it drop out via gravity flow. Collect sample and use in assay of choice.

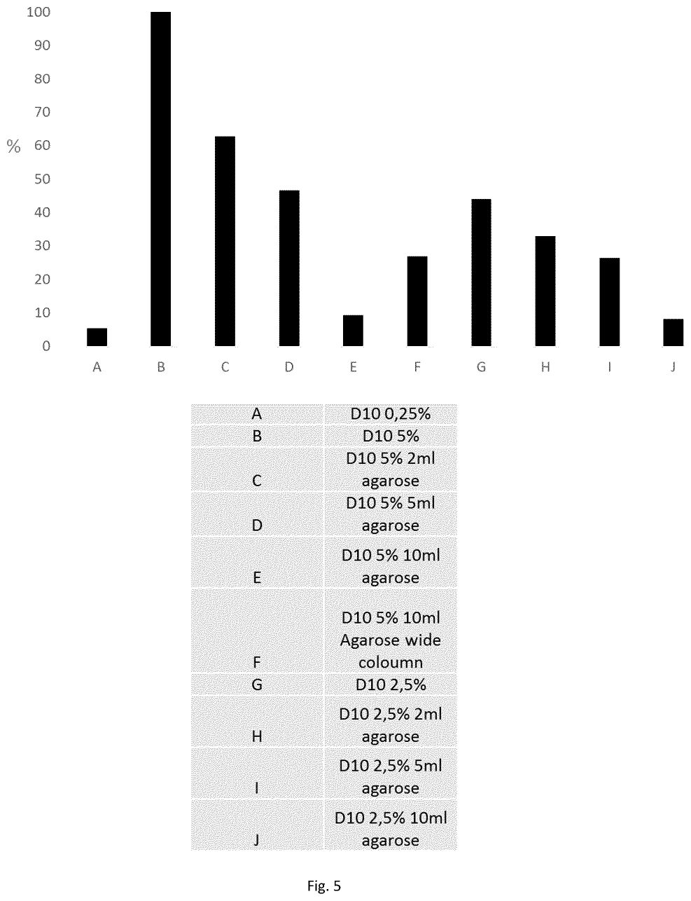

In the experiment relating to FIG. 5, 5 ml of 5%/2.5% DIBMA10 solution was run over a column with different volumes of CDγ coupled agarose beads resulting in a reduction capacity of up to 90% (5 ml pure agarose beads over 10 cm bed height). Bed volume and column diameter play an important role in DIBMA10 binding capacity. 5% DIBMA10 set as 100%.

### Comparative Example: Depletion With Superose 6

Comparison of free copolymer reduction utilising Cyclodextrin coupled agarose vs Size Exclusion chromatography.

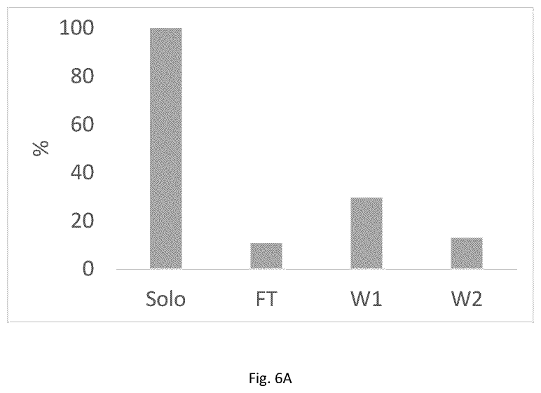

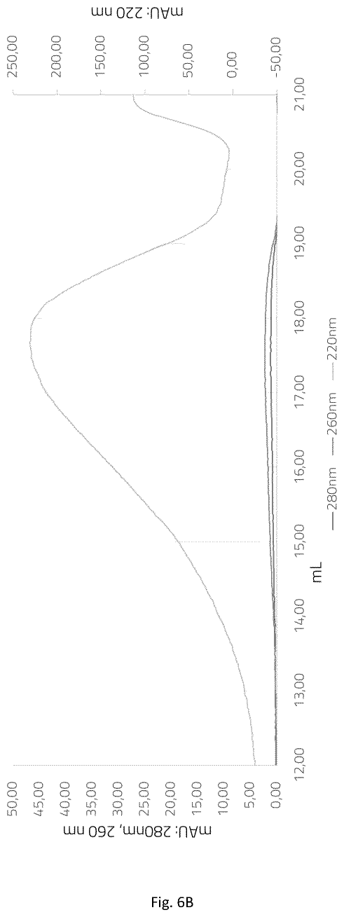

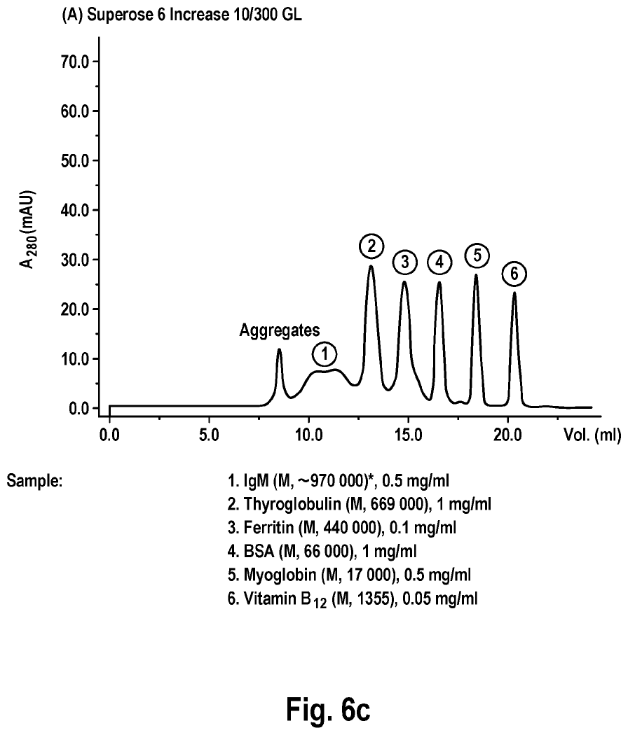

2.5% Ultrasolute Amphipol solution was run over cyclodextrin coupled agarose beads and washed two times with an equal volume (FIG. 6A), showing a 90% decrease of detectable Ultrasolute Amphipol in the flow through (FT). The first wash (W1) shows 30 %, the second wash (W2) 13 % detectable Ultrasolute Amphipol. The copolymer binds to the Cyclodextrin matrix while traveling through the resin then gets washed out over multiple steps. This is not the case if a 2.5% Ultrasolute Amphipol solution is run over a Superose 6 10/300 GL coloumn (Cytiva) (FIG. 6B). The copolymer elutes broadly over a volume of 7.5 ml. This equals a size distribution from ˜700 kDa to ˜17 kDa (comparison FIG. 6C) showing that an extraction of free copolymer via Size Exclusion Chromatography is not possible since a majority of proteins also elutes in a similar manner. On the other hand passing a copolymer solubilized membrane solution over the Cyclodextrin matrix is an easy, fast and reliable way to extract free copolymer from the solution.

### Example 3: Protocol for Removal of Ppolymers from a Cell Lysis Solution Containing Complexed and Solubilized Membrane Proteins

The general description of this protocol is as follows: The solution can contain up to 5% of detergent and amphiphilic polymers. The used distilled water, buffer and protein solution can either flow gravimetric through the column or can be gently pushed through the column by manually or automized applying pressure.
1. Prepare a spin column, dropping tube or comparable column with a frit and filled with 2 mL (50% suspension) of partial or fully hydrophobic functionalized resin (e.g. cyclodextrin agarose or butyl agarose).
2. Wash the compacted agarose with three resin volumes of distilled water and three resin volumes protein buffer (e.g. 150 mM NaCl, 20 mM HEPES, pH 7.5) for equilibration.
3. Load the column with 500 μL cell lysate containing the desired protein nanodisc complexes and the excess of polymer and let the solution fully enter the resin. Discard the flow through of resin loading.
4. Add 1.5 mL protein buffer for elution of the solubilized membrane proteins and collect the flow through which contain the desired protein nanodisc complexes.

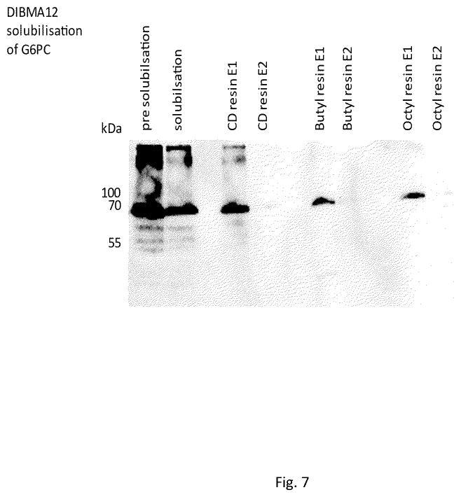

FIG. 7 describes the results from the removal of polymers from a cell lysis solution containing complexed and solubilized membrane proteins. The solution can contain up to 5% of AASTY copolymer. The used distilled water, buffer and protein solution can either flow gravimetric through the column or can be gently pushed through the column by manually or automized applying pressure.
1. Prepare a spin column, dropping tube or comparable column with a frit and filled with 2 mL (50% suspension) of partial or fully hydrophobic functionalized resin (e.g. cyclodextrin agarose or butyl agarose).
2. Wash the compacted agarose with three resin volumes of distilled water and three resin volumes protein buffer (e.g. 150 mM NaCl, 20 mM HEPES, pH 7.5) for equilibration.
3. Load the column with 500 μL cell lysate containing the desired protein nanodisc complexes and the excess of polymer and let the solution fully enter the resin. Discard the flow through of resin loading.
4. Add 1.5 mL protein buffer for elution of the solubilized membrane proteins and collect the flow through which contain the desired protein nanodisc complexes.

1. A method for removing free polymers present in a solution containing a hydrophobic protein, wherein the method comprises:
(a) contacting the solution containing the free polymers not bound to the hydrophobic protein, with a macrocycle and/or a hydrophobic particle so that the free polymers bind to the macrocycle and/or hydrophobic particle to obtain a complex of said polymers and the macrocycle and/or hydrophobic particle, and
(b) removing the complex of polymer and the macrocycle and/or hydrophobic particle from the solution.

2. The method according to claim 1, wherein the hydrophobic protein is a membrane protein.

3. The method according to claim 2, wherein the membrane protein is a membrane associated protein or an integral membrane protein.

4. The method according to claim 2, wherein the membrane protein is selected from the group consisting of membrane receptor proteins, membrane enzymes, cell adhesion proteins, and transporter proteins, such as ABC transporters, ion channel proteins, water channel proteins (aquaporins), membrane-based ATPases, SLC transporters.

5. The method according to claim 1, wherein the macrocycle is selected from the group consisting of cyclodextrins, calixarenes, curcurbituryls and pillararenes, and the hydrophobic particle is butyl agarose,

6. The method according to claim 5, wherein the cyclodextrin is selected from the group consisting of α-cyclodextrin, β-cyclodextrin, γ-cyclodextrin and δ-cyclodextrin or a mixture of at least two of said cyclodextrins.

7. The method according to claim 5, wherein the macrocycle is provided on magnetic beads or agarose beads, or as particles comprising of cross-linked cyclodextrin.

8. The method according to claim 1, wherein the macrocycle and/or hydrophobic particle is washed with a solvent, which is the solvent of the solution of the polymer, before step (a) is carried out.

9. The method according to claim 1, wherein step (a) and step (b) are repeated at least once.

10. The method according to claim 1, wherein the polymer is a polymer that can solubilize and stabilize membrane proteins and GPCRs.

11. The method according to claim 10, wherein said polymer has hydrophilic groups, such as COOH, maleimide, OH, amines, ammonium salts, zwitter ions like phosphocholines, and hydrophobic groups, such as polymerized styrene groups, polymerized diisobutylene groups, or linear C1 to C16 aliphatic group, branched C1 to C16 aliphatic group, and cyclic C5 to C12 aliphatic group or C5 to C12 aromatic group.

12. The method according to claim 10, wherein the molecular weight of the polymer is 1900 to 20000.

13. The method according to claim 10, wherein the polymer is selected from the group consisting of diisobutylene/maleic acid copolymer, styrene maleic acid copolymer, (acrylic acid-co-styrene) copolymer and polyacrylic acid, partially coupled to amides by cycloalkyl amines or cycloalkyl alkylamines.

14. The method according to claim 10, wherein step (a) is carried out 1 minute or less.

15. Use of macrocycles and/or hydrophobic particles in the method according to claim 1.

16. A composition comprising macrocycles bound on a carrier for carrying out the method according to claim 1.

17. A kit for carrying out the method of claim 1, containing:
a macrocycle and/or a hydrophobic particle, and
instructions for carrying out the method for removing the polymer.
